(19) United States
(12) Patent Application Publication
(10) Pub. No.: US 2023/0218630 A1
(43) Pub. Date: Jul. 13, 2023

(54) COMBINATION OF ALCAFTADINE AND A CORTICOSTEROID

(71) Applicant: ALKEM LABORATORIES LIMITED, Mumbai (IN)

(72) Inventors: Ulhas DHUPPAD, Nashik (IN); Akhilesh SHARMA, Mumbai (IN); Babasaheb AWARE, Taluka and District Beed (IN); Viraj SHAH, Dist-Dhule (IN); Amol AIWALE, Wakhari, Pandharpur (IN)

(21) Appl. No.: 17/997,656

(22) PCT Filed: Jun. 14, 2021
(86) PCT No.: PCT/IB2021/055222

§ 371 Date: Oct. 31, 2022

(30) Foreign Application Priority Data
Jun. 15, 2020 (IN) 201921053286

(51) Int. Cl. A61K 31/55; A61K 31/56; A61K 9/00; A61K 47/18; A61K 47/02; A61K 9/10

(52) U.S. Cl. CPC A61K 31/55; A61K 31/56; A61K 9/0043; A61K 47/183; A61K 47/02; A61K 9/10

(57) ABSTRACT

The present invention relates to a topical pharmaceutical composition comprising alcaftadine or a pharmaceutically acceptable salt thereof and a corticosteroid or a pharmaceutically acceptable salt thereof, methods of treating allergic rhinitis, allergic rhino-conjunctivitis, or symptoms thereof (such as nasal congestion) with a combination of alcaftadine or a pharmaceutically acceptable salt thereof and a corticosteroid or a pharmaceutically acceptable salt thereof, and methods of preparing the topical composition.

This application is a national stage of International Patent Application No. PCT/IB2021/055222, filed Jun. 14, 2021, which claims priority to Indian Patent Application No. 201921053286 filed Jun. 15, 2020, the entire contents of which are hereby incorporated by reference herein.

### FIELD OF THE INVENTION

The present invention relates to a topical pharmaceutical composition comprising alcaftadine or a pharmaceutically acceptable salt thereof and a corticosteroid or a pharmaceutically acceptable salt thereof, methods of treating allergic rhinitis, allergic rhino-conjunctivitis, or symptoms thereof (such as nasal congestion) with a combination of alcaftadine or a pharmaceutically acceptable salt thereof and a corticosteroid or a pharmaceutically acceptable salt thereof, and methods of preparing the topical composition.

### BACKGROUND OF THE INVENTION

Allergic rhinitis is among the most common disease affecting globally. Allergic rhinitis persists throughout the life. It has been reported that allergic rhinitis self-reported prevalence ranges from 2% to 25% in children and 1% to greater than 40% in adults. Symptoms of allergic rhinitis include sneezing, rhinorrhea, nasal itching and nasal congestion. Ocular symptoms are also common. Allergic rhino-conjunctivitis is associated with itching and redness of the eyes and tearing.

Allergic rhinitis is characterized by inflammation of the nasal mucous membranes because of a complex response to nasal allergen exposure. The levels of histamine are raised in allergic rhinitis. Allergic rhinitis is characterised by sensitization-formation and expression of antigen specific IgE, followed by inflammation in two phases viz. early and late phase. Mast cells appear to be activated during the early reaction and basophils during the late reaction. The early phase develops in 30 minutes and disappears, characterized by sneezing and rhinorrhea. The early phase response involves cross linking of IgE molecules leading to degranulation of mast cells and release of mediators such as histamine, tryptase, prostaglandins, chymase, kinins, heparins and leukotrienes. The late reaction is inflammatory in nature and shows nasal obstruction approximately six hours after exposure to allergens and subsides slowly. The late phase response is characterized by an inflammatory cellular influx of T lymphocytes, basophils and eosinophils. The late phase response involves mediators released by cells including leukotrienes, kinins, histamine, cytokines and chemokines. These mediators lead to the symptoms of rhinorhhea, nasal congestion, sneezing, itching, redness of nose, watery eyes, swelling of rhino-pharyngeal region, increase in ear pressure and postnasal drip.

Nasal congestion is one of the most common symptoms encountered in primary care and specialist clinics, and it is the symptom that is most bothersome to patients. Mucosal inflammation is responsible for many of the distinct and interrelated factors that contribute to congestion, including increased venous engorgement, elevated nasal secretions, and tissue swelling or edema.

Research shows that more than 60% of patients with allergic rhinitis are not satisfied with their current treatment, particularly due to lack of efficacy (Bousquet et al., J Allergy Clin Immunol., 2009 September, 124(3):428-33).

U.S. Pat. No. 5,468,743 discloses alcaftadine and methods of treating allergic conditions.

U.S. Pat. No. 8,664,215 discloses an ophthalmic alcaftadine composition and methods of treating or preventing ocular allergy.

CN 102283849 discloses a combination of alcaftadine and pseudoephedrine as well as a combination of alcaftadine, pseudoephedrine and acetaminophen for the relief of symptoms related to allergic rhinitis and allergic conjunctivitis.

U.S. Pat. No. 5,164,194 discloses a medicament for nasal use or for use in the eye which contains as an active ingredient azelastine.

US 2009/0324699 discloses a pharmaceutical composition comprising a corticosteroid and an antihistamine, a polar lipid liposome, and a pharmaceutically acceptable aqueous carrier. However, such compositions involve use of a complex process for manufacturing such liposomes.

WO 2019/022225 describes a preservative-free aqueous pharmaceutical composition containing alcaftadine or a salt thereof at a concentration of more than 0.15% w/v.

There exists a need for intranasal compositions which provide improved relief including a faster onset of action compared to the currently available therapies.

### SUMMARY OF INVENTION

The present invention relates to a topical pharmaceutical composition useful for the treatment of allergic rhinitis, allergic rhino-conjunctivitis, and nasal congestion comprising alcaftadine or a pharmaceutically acceptable salt thereof and a corticosteroid or a pharmaceutically acceptable salt thereof. The topical composition is preferably suitable for intranasal or ocular administration. The topical composition optionally includes one or more pharmaceutically acceptable excipients. The composition imparts enhanced mucoadhesion to the nasal mucosa, optimal penetration in the nasal mucosa, and minimal nasal irritation. The topical composition also has improved organoleptic properties that provide better patient compliance and treatment outcomes than other antihistamine products which provide an unpleasant taste, such as azelastine products. In one embodiment, the topical composition has a faster onset of relief compared to other nasally administered antihistamines, such as olopatadine, and corticosteroids. In one embodiment, the topical composition, when intranasally administered, has an onset of action in less than 15 or 10 minutes. In another embodiment, the composition is devoid of an unpleasant taste.

In one embodiment, the topical pharmaceutical composition (e.g., a nasal pharmaceutical composition) is an aqueous pharmaceutical composition comprising (i) about 0.05 to about 5% w/w alcaftadine or a pharmaceutically acceptable salt thereof (such as alcaftadine) (for example, about 0.125 to about 0.75% w/w alcaftadine, such as 0.125, 0.25, 0.35, 0.45, 0.5, 0.6, and 0.75% w/w), (ii) about 0.01 to about 5% w/w fluticasone furoate or fluticasone propionate (e.g., about 0.036% w/w fluticasone propionate or about 0.02% w/w fluticasone furoate), (iii) optionally about 0.01 to about 1.0% w/w suspending agent (such as hydroxypropylmethylcellulose, povidone, sodium carboxymethylcellulose, or hydroxyethyl cellulose), (iv) about 0.068 to about 6.8% w/w of sodium chloride, (v) about 0.0019 to about 0.19% w/w monobasic sodium phosphate, (v) about 0.005 to about 0.5% w/w disodium edetate (e.g., disodium edetate dihydrate), (vi) about 0.0025 to about 0.25% w/w benzalkonium chloride, (vii) about 0.001 to about 0.5% w/w polysorbate 80, and (viii) about 0.5 to about 15% w/w of a co-spray dried combination of microcrystalline cellulose and sodium carboxymethylcellulose (e.g., where the combination includes 11.3 to 18.8% sodium carboxymethylcellulose). The composition may include amounts of sodium hydroxide and/or hydrochloric acid to achieve the desired pH, such as a pH of 6.0 to 7.5, 6.5 to 7.0 or 6.3 to 7.3 (e.g., 6.7 to 7.3).

In another embodiment, the topical pharmaceutical composition (e.g., a nasal pharmaceutical composition) is an aqueous pharmaceutical composition comprising (i) about 0.125% w/w alcaftadine, (ii) about 0.036% w/w fluticasone propionate or about 0.02% w/w fluticasone furoate, (iii) optionally about 0.1% w/w suspending agent (such as hydroxypropylmethylcellulose, povidone, sodium carboxymethylcellulose, or hydroxyethyl cellulose), (iv) about 0.068 to about 6.8% w/w of sodium chloride, (v) about 0.0019 to about 0.19% w/w monobasic sodium phosphate, (vi) about 0.005 to about 0.5% w/w disodium edetate (e.g., disodium edetate dihydrate), (vii) about 0.0025 to about 0.25% w/w benzalkonium chloride, (viii) about 0.001 to about 0.5% w/w polysorbate 80, and (ix) about 0.5 to about 15% w/w of a co-spray dried combination of microcrystalline cellulose and sodium carboxymethylcellulose (e.g., where the combination includes 11.3 to 18.8% sodium carboxymethylcellulose). The composition may include amounts of sodium hydroxide and/or hydrochloric acid to achieve the desired pH, such as a pH of 6.0 to 7.5, 6.5 to 7.0 or 6.3 to 7.3 (e.g., 6.7 to 7.3).

In yet another embodiment, the topical pharmaceutical composition (e.g., a nasal pharmaceutical composition) is an aqueous pharmaceutical composition comprising (i) about 0.125% w/w alcaftadine, (ii) about 0.036% w/w fluticasone propionate or about 0.02% w/w fluticasone furoate, (iii) about 0.68% w/w of sodium chloride, (iv) about 0.019% w/w monobasic sodium phosphate, (v) about 0.05% w/w disodium edetate (e.g., disodium edetate dihydrate), (vi) about 0.0125% w/w benzalkonium chloride, (vii) about 2.1% glycerine, (viii) about 0.003% w/w polysorbate 80, and (ix) about 3.64% w/w of a co-spray dried combination of microcrystalline cellulose and sodium carboxymethylcellulose (e.g., where the combination includes 11.3 to 18.8% sodium carboxymethylcellulose). The composition may include amounts of sodium hydroxide and/or hydrochloric acid to achieve the desired pH, such as a pH of 6.0 to 7.5, 6.5 to 7.0 or 6.3 to 7.3 (e.g., 6.7 to 7.3).

The topical pharmaceutical compositions described herein are stable. In one embodiment, the topical composition, after 24 hours, 3 or 6 months storage at 25° C. and 60% relative humidity or 40° C. and 75% relative humidity has at least 90, 95, or 98% of the initial amount of each active ingredient present. In another embodiment, the amount of any single individual impurity (of an active ingredient) is no more than 0.5%, 0.2%, 0.1% or 0.05% by weight, based on the amount of the active ingredient present. In yet another embodiment, the amount of total impurities (of an active ingredient) is no more than 3%, 2%, 1%, 0.5%, 0.3%, or 0.2% by weight, based on the amount of the active ingredient present.

Another embodiment is a method of treating allergic rhinitis, allergic rhino-conjunctivitis, or symptoms thereof (such as nasal congestion) in a patient in need thereof by topically administering an effective amount of alcaftadine or a pharmaceutically acceptable salt thereof and a corticosteroid or a pharmaceutically acceptable salt thereof. In one embodiment, the alcatfadine and corticosteroid are intranasally administered. In another embodiment, the alcatfadine and corticosteroid are ocularly administered. In one preferred embodiment, the method includes administering (e.g., intranasally) the topical composition of the present invention. In one embodiment, the patient suffers from allergic rhinitis. In another embodiment, the patient suffers from seasonal allergic rhinitis. In yet another embodiment, the patient suffers from perennial allergic rhinitis. In yet another embodiment, the patient suffers from moderate to severe seasonal allergic rhinitis. In yet another embodiment, the patient suffers from moderate to severe perennial allergic rhinitis.

Yet another embodiment is a method of treating allergic rhinitis, allergic rhino-conjunctivitis, or a symptom thereof (such as nasal congestion) in a patient in need thereof by topically administering an effective amount of the topical composition of the present invention. In one embodiment, the topical composition is intranasally administered. In another embodiment, the topical composition is ocularly administered. In one embodiment, the patient suffers from allergic rhinitis. In another embodiment, the patient suffers from seasonal allergic rhinitis. In yet another embodiment, the patient suffers from perennial allergic rhinitis. In yet another embodiment, the patient suffers from moderate to severe seasonal allergic rhinitis. In yet another embodiment, the patient suffers from moderate to severe perennial allergic rhinitis

Yet another embodiment is a method of reducing the use of nasal decongestants by topically administering an effective amount of alcaftadine or a pharmaceutically acceptable salt thereof and a corticosteroid or a pharmaceutically acceptable salt thereof. Preferably, the topical pharmaceutical composition of the present invention is intranasally administered.

Yet another embodiment is a method of inhibiting, suppressing, or preventing nasal polyps in a patient in need thereof by topically administering an effective amount of alcaftadine or a pharmaceutically acceptable salt thereof and a corticosteroid or a pharmaceutically acceptable salt thereof. In one embodiment, the alcatfadine and corticosteroid are intranasally administered. In another embodiment, the alcatfadine and corticosteroid are ocularly administered. In one preferred embodiment, the method includes administering (e.g., intranasally) the topical composition of the present invention.

In the methods described herein, the alcaftadine and corticosteroid may be administered simultaneously, separately or sequentially.

The methods and topical compositions described herein can result in better patient compliance than other treatments for allergic rhinitis as the present inventors discovered that alcaftadine, when intranasally administered does not result in an unpleasant taste as with the antihistamine azelastine.

Yet another embodiment is a process for the preparation of a topical pharmaceutical composition (such as those described herein) comprising the steps of (i) dissolving a pH adjusting agent (e.g., hydrochloric acid), a tonicity adjustment agent (e.g., sodium chloride), and alcaftadine in water to form a first active phase solution, (ii) mixing a surfactant and cosolvent (e.g., glycerin), and a corticosteroid (e.g., fluticasone propionate or fluticasone furoate) in water to form a first dispersion, (iii) dispersing (and optionally homogenizing) a suspending agent (e.g., a co-spray dried combination of microcrystalline cellulose and carboxymethylcellulose sodium) in water to form a second dispersion, (iv) adding the first dispersion to the second dispersion to form a second active phase solution, (v) adding a chelating agent (e.g., disodium edetate such as disodium edetate dihydrate) and a buffering agent (e.g., monobasic sodium phosphate) to water to form a third solution, (vi) adding the first active phase solution and the third solution from step (v) to the second active phase solution (e.g., under stirring) to obtain a dispersion, (vii) adding a preservative (e.g., benzalkonium chloride) to the solution prepared in step (vi), (viii) optionally, adjusting the pH of the solution from step (vii) using sodium hydroxide (e.g., to a pH of 6.0 to 7.5, 6.5 to 7.0 or 6.3 to 7.3), and (ix) optionally, adding water (e.g., purified water) to the solution of step (viii) to obtain a desired volume and/or concentration for each component.

### DETAILED DESCRIPTION OF THE INVENTION

The inventors of the present invention have found that alcaftadine or a pharmaceutically acceptable salt thereof can be combined with a corticosteroid or a pharmaceutically acceptable salt thereof to provide an advantageous composition for treatment of allergic rhinitis, allergic rhino-conjunctivitis, and symptoms thereof (such as nasal congestion). Without being bound by any particular theory, alcaftadine due to its H₄ receptor antagonist activity which is expected to upregulate lipocortin-1, is believed to potentiate the activity of corticosteroids resulting in a synergistic combination and permit reduction in the dosage of the corticosteroid without a loss in clinical activity or increase clinical activity at the same dose.

The topical composition for intranasal administration (such as a combination of alcaftadine and fluticasone or an ester thereof) provides a rapid onset of action, for instance within 0.15, 0.1, 0.08, or 0.05 hours. The topical composition for intranasal administration provides improved relief from nasal itching and nasal decongestion. Furthermore, the topical composition has fewer side effects. Other antihistaminic agents have central nervous system adverse effects due to inverse agonism at H₁-receptors, inhibition of neurotransmission in histaminergic neurons, and impairment of alertness, cognition, learning, and memory that is not necessarily associated with sedation, drowsiness, fatigue, or somnolence. Intranasal administration of alcaftadine provides a local effect without systemic absorption and therefore without CNS side effects. For instance, an intranasal composition of alcaftadine with a corticosteroid (such as fluticasone or an ester thereof) is associated with fewer adverse reactions such as headache and epistaxis as compared to olapatadine and azelastine nasal spray.

One embodiment is a nasal composition of alcaftadine in fixed combination with fluticasone or an ester thereof (such as fluticasone propionate or fluticasone furoate), characterized in that the composition has a faster onset of relief compared to fixed combinations of other anti-histaminics (such as azelstine and olopatadine) and corticosteroids. In one preferred embodiment, the composition provides a rapid onset of action with a Tₘₐₓ for alcaftadine of about 0.25 hours or less (such as 0.2, 0.15, 0.1, 0.08, or 0.05 hours).

In a further embodiment, the present invention provides a pharmaceutical composition of alcaftadine in fixed combination with fluticasone or an ester thereof (such as fluticasone propionate or fluticasone furoate) for the treatment of symptoms associated with allergic rhinitis such as sneezing, nasal itching, nasal inflammation, nasal irritation, rhinorrhea, nasal pruritus and nasal congestion. The composition is an effective nasal decongestant. As a result, patients can reduce their concomitant use of other nasal decongestants.

The topical composition of the present invention can be administered in the nostril(s) or to the eyes once or twice a day.

### Alcaftadine

Alcaftadine (6,11-dihydro-11-(1-methyl-4-piperidinylidene)-5H-imidazo[2,1-b][3]benzazepine-3-carboxaldehyde is an anti-allergic therapeutic agent that has inverse agonist effects on H₁, H₂, and H₄ receptors, as well as mast cell-stabilizing effects.

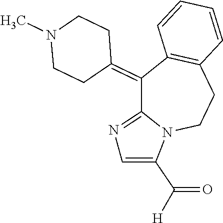 [SMILES: CN1CCC(=C2c3ccccc3CCn3c(C=O)cnc32)CC1]

H₂ receptors play a vital role in nasal congestion. H₄ receptors influence the inflammatory response (eosinophils, T cells, dendritic cells, basophils, mast cells, and sensory nerve cells). Alcaftadine has higher binding affinity to H₁ and H₂ receptors compared to other antihistamines namely azelastine and olapatadine, and demonstrates higher efficacy in controlling total nasal symptoms, including congestion. H₁ and H₂ receptor signaling contributes to pruritus, redness of nose, cytokine secretion, fibroblast proliferation, adhesion molecule expression, microvascular permeability and production of procollagens. Estelle et al., J Allergy Clin Immunol, 2011, 128:1139-50. H₄ receptor signaling has been shown to affect cytokine and chemokine release, chemotaxis, and adhesion molecule expression in allergic rhinitis in experimental allergic rhinitis model studies. Hanuskova et al., Open Journal of Molecular and Integrative Physiology, 2013, 3:6-14.

### Corticosteroids

The methods and compositions described herein include one or more corticosteroids. Suitable corticosteroids include, but are not limited to, alclometasone, beclometasone, betametasone, budesonide, ciclesonide, clobetasol, clobetasone, deflazacort, deprodone, dexamethasone, diflucortolone, fluocinolone, etiprednol, flunisolide, fluocinonide, fluocortolone, fluprednidene, flurometholone, fluticasone, halcinonide, hydrocortisone, KSR-592, loteprednol, methylprednisolone, mometasone, prednisolone, rimexolone, triamcinolone, esters thereof, and pharmaceutically acceptable salts thereof. In one embodiment, the corticosteroids are selected from beclomethasone, mometasone and esters thereof (such as mometasone furoate), fluticasone and esters thereof (such as fluticasone furoate and fluticasone propionate), budesonide, ciclesonide, and pharmaceutically acceptable salts thereof. In yet another embodiment, the corticosteroid is a glucocorticoid.

In one preferred embodiment, when the topical composition is a nasal composition or the corticosteroid is to be administered intranasally, the corticosteroids are selected from beclomethasone, mometasone and esters thereof (such as mometasone furoate), fluticasone and esters thereof (such as fluticasone furoate and fluticasone propionate), budesonide, ciclesonide, and pharmaceutically acceptable salts thereof.

In a more preferred embodiment, the corticosteroid is fluticasone, an ester thereof (such as fluticasone furoate, fluticasone propionate, and fluticasone valerate), or a pharmaceutically acceptable salt thereof. Any ester of fluticasone can be used in the topical composition and methods of the present invention. Preferably, the ester of fluticasone is selected from fluticasone propionate, fluticasone furoate, and fluticasone valerate. Fluticasone is currently commercially available in the form of fluticasone furoate and fluticasone propionate.

Fluticasone propionate is a corticosteroid having the chemical name S-(fluoromethyl) 6α,9-difluoro-11β,17-dihydroxy-16α-methyl-3-oxoandrosta-1,4-diene-17β-carbothioate, 17-propionate. Fluticasone propionate exhibits high lipophilicity, high selectivity and affinity for the glucocorticoid receptor, low oral systemic absorption, and rapid metabolic clearance. Fluticasone propionate has the chemical formula (I)

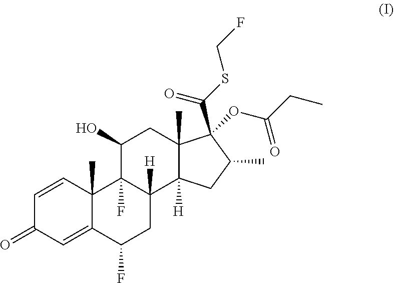

Fluticasone Furoate has the Chemical Formula (II)

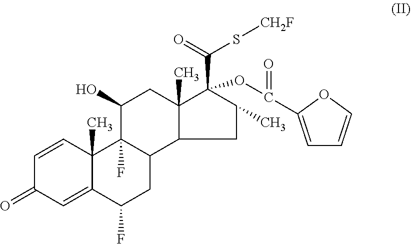

### Dosage Form

The present invention provides alcaftadine and a corticosteroid (e.g., fluticasone propionate or fluticasone furoate) in unionized form at the site of action, thereby improving their permeability through the nasal mucosa. The spray can, for example, be formed by the use of a conventional spray-squeeze bottle or a pump vaporizer. In addition, it is also possible to use compressed gas aerosols.

The topical composition may provide simultaneous or sequential release of the alcaftadine and corticosteroid. The topical composition may be a nasal composition, such as a nasal solution, nasal suspension, nasal powder, nasal spray, nasal aerosol, nasal drops, nasal ointment, nasal inhalation or nasal gel.

In one embodiment, the topical composition comprises an effective amount of alcaftadine in the range of 0.05% to 5% w/w in combination with an effective amount of fluticasone or an ester thereof (such as fluticasone propionate or fluticasone furoate) in the range of 0.01% to 5% w/w. For example, the topical composition may include 0.1% w/w to 4% w/w, such as 0.1% w/w to 3% w/w, 0.1% w/w to 2% w/w, or 0.1 w/w % to 1% w/w (for example, 0.125% w/w, 0.25% w/w or 0.50% w/w) of alcaftadine, based upon 100% total weight of the composition. In one embodiment, the alcaftadine is present in dissolved form in the composition. The composition may include 0.01 to 0.1% w/w of a corticosteroid (such as fluticasone propionate or fluticasone furoate), for example, about 0.036% w/w fluticasone propionate or about 0.02% w/w fluticasone furoate.

In one embodiment, the alcaftadine or pharmaceutically acceptable salt thereof (e.g., alcaftadine in free form) and fluticasone propionate are present in a weight ratio of about 5:1 to about 1:200 or from about 1:1 to about 1:150, and preferably from about 1:2 to about 1:110.

In one embodiment, the alcaftadine or pharmaceutically acceptable salt thereof (e.g., alcaftadine in free form) and fluticasone furoate are present in a weight ratio of about 5:1 to about 1:300 or from about 3:1 to about 1:250, and preferably from about 1:1 to about 1:200.

In another embodiment, the alcaftadine or pharmaceutically acceptable salt thereof (e.g., alcaftadine in free form) and fluticasone propionate are present in a molar ratio of about 5:1 to about 1:250 or from about 3:1 to about 1:200 and preferably from about 1:1 to about 1:180.

In one embodiment, the alcaftadine or pharmaceutically acceptable salt thereof (e.g., alcaftadine in free form) and fluticasone furoate are present in a molar ratio of about 5:1 to about 1:500 or from about 2:1 to about 1:400 and preferably from about 1:0.7 to about 1:360.

In one embodiment, the topical composition is a suspension. For example, the alcaftadine is in dissolved form and the corticosteroid (such as fluticasone or an ester thereof, for example, fluticasone propionate or fluticasone furoate) is in particulate form in the topical composition. The corticosteroid (such as fluticasone propionate or fluticasone furoate) can have a d50 of from about 1 to about 100 μm, such as from about 1 to about 10 μm. In one embodiment, the corticosteroid can have a d90 of from about 1 to about 100 μm, such as from about 3 to about 15 μm.

The topical composition may include one or more pharmaceutically acceptable excipients. Suitable excipients include, but are not limited to, mucoadhesive agents, buffering agents, osmotic agents or tonicity adjustment agents, chelating agents, permeation enhancers, surfactants, pH adjusting agents, suspending agents, thickening agents (or viscosity modifiers), preservatives, solubilizers, and vehicles (such as solvents and cosolvents).

The topical composition for intranasal administration can include a mucoadhesive agent to provide better adherence to nasal mucosa and improve retention of alcaftadine and the corticosteroid on the nasal mucosa. Suitable mucoadhesive agents include, but are not limited to, cellulose derivatives (such as hydroxypropylmethyl cellulose, hydroxyethylcellulose, and carboxymethylcellulose sodium), povidone, chitosan, poloxamers (Pluronic®) and natural gums (such as guar gum and xanthan gum). Mucoadhesive agents are often temperature dependent which upon applying or spraying in the nasal cavity form a gel on the nasal mucosa. The gel provides longer contact and retention time for the alcaftadine and corticosteroid on the nasal mucosa thereby providing extended relief from nasal decongestion. The mucoadhesive agents may be used in an amount from 0.01% to 10% by weight of the total composition, preferably 0.01% w/w to 1% w/w (based on the composition), such as 0.01% w/w to 0.9% w/w, 0.01% w/w to 0.7% w/w, 0.01% w/w to 0.5% w/w, 0.01% w/w to 0.3% w/w, or 0.01% w/w to 0.1% w/w. The mucoadhesive agents may be temperature dependent which upon applying or spraying in the nasal cavity form a gel on the nasal mucosa. The gel provides longer contact and retention time for the alcaftadine and corticosteroid on the nasal mucosa thereby providing extended relief from nasal decongestion.

In one embodiment, the composition has a sufficient amount of the mucoadhesive agent to provide the composition with a contact angle less than 109°, 108°, 107°, 106°, 105°, 104°, or 103°.

Suitable buffering agents include, but are not limited to, monobasic sodium phosphate, disodium hydrogen phosphate, dibasic sodium phosphate, tribasic sodium phosphate, and dibasic potassium phosphate. Buffering agents may be used in an amount from 0.005% to 2% by weight of the total composition, such as 0.0009% w/w to 0.19% w/w, 0.0009% w/w to 0.1% w/w, 0.009% w/w to 0.1% w/w, 0.007% w/w to 0.1% w/w, or 0.005% w/w to 0.1% w/w.

Osmotic agents or tonicity adjustment agents refer to agents that are specifically added to the composition to increase the solute level in the composition and contribute to achieving isotonicity of the topical composition. Tonicity is the ‘effective osmolality’ and is equal to the sum of the concentrations of the solutes which have the capacity to exert an osmotic force across the membrane. Suitable tonicity adjustment agents include, but are not limited to, sodium chloride, dextrose (e.g., dextrose USP), glycerine (e.g., glycerin USP), mannitol (e.g., mannitol USP), and potassium chloride (e.g., potassium chloride USP). In one embodiment, the topical composition for intranasal administration contains sodium chloride in an amount sufficient to cause the composition to have a nasally acceptable osmolality, preferably 50-700 mOsmol/kg. The tonicity adjustment agent may be used in an amount from 0.050% to 7% by weight of the total composition. In a preferred embodiment, the nasal pharmaceutical composition contains 0.068% w/w to 6.8% w/w of sodium chloride, based on the composition, such as 0.068% w/w to 5.8% w/w, 0.068% w/w to 4.8% w/w, 0.058% w/w to 3.8% w/w, 0.058% w/w to 2.8% w/w, or 0.058% w/w to 1.8% w/w.

Suitable chelating agents include, but are not limited to, edetate disodium, diethylene-triamine-pentaacetic acid (DTPA), iminodisuccinic acid, and ethylenediamine disuccinic acid. The chelating agent may be used in an amount from 0.005% to 0.5% by weight of the total composition. In one embodiment, the composition includes edetate disodium dihydrate in the range of 0.005% w/w to 0.5% w/w, based on the composition, such as 0.005% to 0.4% w/w, 0.005% w/w to 0.3% w/w, 0.005% w/w to 0.2% w/w, or 0.005% w/w to 0.1% w/w.

The permeation enhancer can enhance the permeation of alcaftadine and/or corticosteroid through the nasal mucosa. The permeation enhancer can be a hydroxyl group-containing compound. Non-limiting examples of hydroxyl group-containing compounds that may be used as permeation enhancers include alcohols (such as ethanol), diols (such as propylene glycol (also known as 1,2-propanediol), 1,3-propanediol, butylene glycol (including 1,3-butanediol, 1,2-butanediol, 2,3-butanediol, and 1,4 butanediol), hexylene glycol, dipropylene glycol, 1,5-pentanediol, 1,2-pentanediol, 1,8-octanediol, etohexadiol, p-menthane-3,8 diol, and 2-methyl-2,4-pentanediol), triols (such as glycerin), polyols (such as suitable polymers containing multiple hydroxyl groups) (including polyethylene glycols (PEGs), polypropylene glycols, polysorbates, and sorbitan esters, and suitable sugar alcohols), cyclitols (such as pinitol, insoitol), cyclic diols (such as cyclohexane diol), aromatic diols (such as hydroquinone, bisphenol A, resorcinol and catechol) or any combination thereof. Other permeation enhancer include, but are not limited to, bile salts, Vitamin E TPGS, Alkyl Maltosides, non-ionic, anionic or amphoteric surfactants having HLB value 8-14 or combination thereof. The non-limiting examples of such permeation enhancers are sodium glycocholate, sodium taurocholate, dodecyl maltosides, tridecyl maltoside or tetradecyl maltosides, or any combination thereof. In one embodiment, the permeation enhancer may be present in the topical pharmaceutical composition in an amount from 0.5% to 50% by weight of the total composition, such as 2% w/w, 5% w/w, 7.5% w/w, 10% w/w, 20% w/w, and 40% w/w.

Suitable surfactants (or wetting agents) include non-ionic, cationic, amphoteric, and anionic surfactants. Suitable surfactants include, but are not limited to, sodium lauryl sulfate; polyoxyethylene derivatives of fatty acid partial esters of sorbitol anhydrides such as polysorbates selected from polysorbate 20, polysorbate 40, polysorbate 60, polysorbate 65, polysorbate 80, polysorbate 85, sorbitan fatty acid esters such as sorbitan monolaurate (Span® 20), soribtan monopalmitate (Span® 40), sorbitan monostearate (Span® 60), sorbitan oleate (Span® 80), and sorbitan isostearate (Span® 120); polyethoxylated castor oil; polyethoxylated hydrogenated castor oil, sodium dodecyl sulfate (sodium lauryl sulfate), polyoxyethylene sorbitan, octoxynol, polyoxyl 10 lauryl ether, polyoxyl castor oil, cyclodextrins, lecithin, carboxylates, sulphonates, polygluconates (short-chain starches), and lignin sulfonates. In a preferred embodiment, surfactants may be used in an amount from 0.001 or 0.5% to 60% by weight (such as 0.001 to 0.1% w/w) of the total composition. Surfactants can affect the surface tension of droplets from a delivered nasal spray plume, producing spherical or substantially spherical particles having a narrow droplet size distribution (DSD), as well as the viscosity of a liquid formulation.

Suitable pH adjusting agents include, but are not limited to hydrochloric acid, sodium hydroxide, ammonium hydroxide, magnesium hydroxide, sulphuric acid, phosphoric acid, citric acid, malic acid, and tartaric acid. The pH adjusting agent may be used in an amount sufficient to obtain a pH of about 3 to about 11, such as about 5 to about 9, about 6 to about 8, about 6 to about 7.5, about 6.3 to about 7.3, or about 6.7 to about 7.3.

Suitable suspending agents and thickening agents include, but are not limited to, cellulose derivatives (for example, cellulose ethers) in which the cellulose-hydroxy groups are partially etherified with lower unsaturated aliphatic alcohols and/or lower unsaturated aliphatic oxyalcohols (for example methyl cellulose, carboxymethylcellulose (e.g., sodium carboxymethylcellulose), hydroxypropylmethylcellulose, and hydroxyethyl cellulose), gelatin, polyvinylpyrrolidone (povidone), tragacanth, alginic acid, polyvinyl alcohol, polyacrylic acid, pectin, microcrystalline cellulose which may or may not co-processed with sodium carboxymethylcellulose, and mixtures thereof. Should these substances contain acid groups, the corresponding physiologically acceptable salts may also be used. Thickening and suspending agents can be added to solutions according to the present invention to prevent the topical composition (such as a nasal solution) from flowing out of the nose too quickly and to give the solution a viscosity of about 100 to about 400 cPs. In a preferred embodiment, thickening agents and suspending agents may be used in an amount from 0.1, 0.2, or 0.5% to 50% by weight (such as 0.1 or 0.2% to 5% w/w) of the total composition.

Suitable preservatives include, but are not limited to, benzalkonium chloride, potassium sorbate, methyl paraben, propyl paraben, chlorbutol, chlorocresol, chlorhexidine, sodium benzoate, benzyl alcohol, and propylene glycol. In a preferred embodiment, preservatives may be used in an amount from 0.0025% to 3% by weight of the total composition. In one embodiment, preservatives may be used in an amount from 0.0025% to 2.5% by weight of the total composition. In another embodiment, the composition contains 0.0025% w/w to 0.25% w/w of a preservative, based on the weight of the composition, such as 0.0025% w/w to 0.15% w/w, 0.0025% w/w to 0.1% w/w, or 0.0025% w/w to 0.05% w/w. In one preferred embodiment, the composition includes benzalkonium chloride. In one embodiment, the composition includes from 0.0025% w/w to 2.5% w/w benzalkonium chloride, based on the weight of the composition.

In another embodiment, the topical composition is preservative-free.

Suitable vehicles (such as solvents and cosolvents) and solubilizers include, but are not limited to, purified water, glycols such as propylene glycol, polyethylene glycol, polypropylene glycol, glycol ether, glycerol (glycerin), polyoxyethylene alcohols and polyoxyethylene fatty acid esters. The vehicle or solubilizer may be present in the composition in an amount from 5% w/w to 20% w/w, based on the weight of the composition, such as 5% w/w to 15% w/w, 5% w/w to 10% w/w, or 5% w/w to 7% w/w. Propylene glycol and polyethylene glycol may be used in an amount from 5% w/w to 20% w/w of the total composition. Purified water may be used in an amount to make the formulation 100% by weight of the total composition.

In one embodiment, the topical composition is a stable suspension in which the alcaftadine or pharmaceutically acceptable salt thereof is in dissolved form, the corticosteroid is in particulate form, glycerin, polysorbate (such as polysorbate 80), and a mixture of microcrystalline cellulose and sodium carboxymethylcellulose. The topical composition may include (a) 0.050% to 7% by weight (such as 0.5 to 4% w/w) of glycerin, (b) 0.001 to 0.5% by weight (such as 0.001 to 0.1% w/w) of polysorbate (e.g., polysorbate 80), and (c) 0.1 to 5% (or 0.2 to 5%) by weight of a mixture of microcrystalline cellulose and sodium carboxymethylcellulose. In one preferred embodiment, the mixture of microcrystalline cellulose and sodium carboxymethylcellulose has a viscosity (2.6% solids, 120 seconds) of 50 to 118 cps and contains 11.3-18.8% sodium carboxymethylcellulose.

In one embodiment, the topical composition has an osmolality of 50 to 700 mOsmol/kg, such as 100 to 600 mOsmol/kg or 100 to 500 mOsmol/kg.

In one embodiment, the topical composition (e.g., a nasal solution) has a viscosity of about 100 to about 400 cPs. The viscosity can be measured with a Brookfiled (DV II+ Pro) AD-VS-02, with a ULA spindle and a 10 mL sample at a 100 rpm, ambient temperature, and a 60 second measurement time. The viscosity measurement can be the average of three measurements.

In one embodiment, the pH of the topical composition ranges from about 3 to about 11. In another embodiment, the pH of the topical composition ranges from about 6 to about 8, such as at about 6 to about 7.5 or about 6.7 to 7.3 (such as 6.8, 6.9, 7.0, 7.1, or 7.2). The topical composition may include a buffering agent in an effective amount to maintain the pH at about 3 to about 11, about 6 to about 8, about 6 to about 7.5, or about 6.7 to 7.3 (such as 6.8, 6.9, 7.0, 7.1, or 7.2).

In one embodiment, the composition has a contact angle less than 109°, 108°, 107°, 106°, 105°, 104°, or 103°.

A spray pump's performance can be characterized in terms of its emitted spray pattern, plume geometry, and/or droplet size distribution. These parameters are known to be affected by size and shape of the nozzle, the design of the pump, the size of the metering chamber, and the characteristics of the formulation. The droplet size is influenced by the actuation parameters of the device and the composition being administered. The prevalent median droplet size can be between about 30 and about 200 μm. Spray characterization (e.g., plume geometry, spray pattern, pump delivery, and droplet size distribution (DSD)) of the delivered plume subsequent to spraying may be measured under specified experimental and instrumental conditions by appropriate and validated and/or calibrated analytical procedures known in the art. These include photography, laser diffraction, and impaction systems (such as cascade impaction and next generation impaction (NGI)).

In one embodiment, the topical pharmaceutical compositions described herein has a droplet size distribution at 3 cm with a D10 of no more than 40 μm (e.g., from about 5 to about 30 μm), D50 of no more than 80 μm (e.g., from about 30 to about 200 μm or from about 10 to about 50 μm), a D90 of no more than 180 μm (e.g., from about 40 to about 100 μm), and/or a SPAN of no more than 3 (e.g., from about 1.0 to about 3.0). In one embodiment, the spray content uniformity is from about 85 to about 115%.

In another embodiment, the topical pharmaceutical compositions described herein has a droplet size distribution at 6 cm with a D10 of no more than 40 μm (e.g., from about 5 to about 30 μm), D50 of no more than 80 μm (e.g., from about 30 to about 200 μm or from about 10 to about 50 μm), a D90 of no more than 180 μm (e.g., from about 40 to about 100 μm), and/or a SPAN of no more than 3 (e.g., from about 1.0 to about 3.0). In one embodiment, the spray content uniformity is from about 85 to about 115%.

Evaluation of the spray pattern, spray distance between the nozzle and the collection surface, number of sprays per spray pattern, position and orientation of the collection surface relative to the nozzle, and visualization procedure are defined in terms of ovality and Dₘₐₓ. In one embodiment, the topical pharmaceutical composition of the present invention has a spray pattern at 3 cm with a Dₘₐₓ of no more than 100 mm (e.g., about 20 to about 100 mm) or no more than 50 mm (e.g., about 20 to about 50 mm), a Dₘᵢₙ of no more than 75 mm (e.g., about 15 to about 75 mm) or no more than 25 mm, an ovality of no more than 2.0 (e.g., about 0.5 to about 2.0), and an area of no more than 2000 mm². In yet another embodiment, the nasal pharmaceutical composition of the present invention has a spray pattern at 6 cm with a Dₘₐₓ of no more than 100 mm (e.g., about 30 to about 100 mm), a Dₘᵢₙ of no more than 75 mm (e.g., about 25 to about 75 mm), an ovality of no more than 2.0 (e.g., about 0.5 to about 2.0), and an area of no more than 5000 mm².

In yet another embodiment, the topical pharmaceutical composition provides a droplet size distribution characterized by one or more of the following: (i) a D10 in the range of no more than 40 μm (such as 5-25 μm), wherein 10% of the droplets in the plume have a size less than the D10, (ii) a D50 of no more than 80 μm (e.g., from 10 to 70 μm), wherein 50% of the droplets in the plume have a size less than the D50, (iiii) a D90 of less than 200 μm or no more than 120 μm, wherein 90% of the droplets in the plume have a size less than the D90, (iv) a SPAN of from 1 to 6, such as no more than 3, wherein the SPAN is calculated according to (D90-D10)/D50, or any combination of any of the foregoing.

In a further embodiment of the present invention, the topical composition is in the form of an aerosol or a solution which includes a delivery system, such as a bottle or a pump delivery or a high-density polyethylene container or a PET (polyethylene terephthalate) container or a glass container equipped with a nasal spray pump, metered-dose spray pump, inhaler, with dropper and other forms for intra-nasal usage. The composition can be delivered in a mist of spray droplets or minor droplets to coat the nasal mucosa upon administration. Preferred pumps for use in such products of the invention are metered multi-dose pumps. The selection of the pump is based on the desired dose per spray volume and spray pattern appropriate for topical delivery to the nasal mucosa. The dosage per spray may range from 1 ml to 100 ml, where each spray may deliver 100 μl to 400 μl per spray.

The topical composition can be in the form of an aerosol and include a propellant. The propellant may be a pressurized liquid chlorinated, fluorinated hydrocarbon or mixtures of various chlorinated, fluorinated hydrocarbons as well as propane, butane, isobutene or mixtures of these among themselves or with chlorinated, fluorinated hydrocarbons which are gaseous at atmospheric pressure and room temperature. Hydrofluorocarbons (HFCs), such as HFC 134a, and HFC 227a, can also be used as the propellant, and are preferred for environmental reasons. The topical composition may be stored under pressure. The aerosol container may have a dosage or metering valve which, on actuation, releases a defined amount of the topical composition (e.g., a solution or suspension). The subsequent very sudden vaporization of the propellant tears the solution or suspension of alcaftadine into fine droplets or minute particles which can be sprayed in the nose or which are available for inspiration into the nose. Certain plastic applicators may be used to actuate the valve and to convey the sprayed topical composition into the nose.

In another embodiment, the topical composition is in the form of an insufflatable powder, for example, where the maximum particle size of the composition does not exceed 10 μm. Alcaftadine or its salt and the corticosteroid may be mixed with one or more inert carrier substances or drawn up onto one or more inert carrier substances. Carrier substances which may, for example, be used include sugars (such as glucose, saccharose, lactose and fructose), starches or starch derivatives, oligosaccharides (such as dextrins), cyclodextrins and their derivatives, polyvinylpyrrolidone, alginic acid, tylose, silicic acid, cellulose, cellulose derivatives (for example cellulose ether), sugar alcohols (such as mannitol or sorbitol), calcium carbonate, calcium phosphate, and any combination of any of the foregoing.

### Preparation Method

One embodiment is a process for the preparation of a topical pharmaceutical composition (such as those described herein) comprising the steps of (i) dissolving a pH adjusting agent (e.g., hydrochloric acid), a tonicity adjustment agent (e.g., sodium chloride), and alcaftadine in water to form a first active phase solution, (ii) mixing a suspending agent, a tonicity adjustment agent (e.g., glycerin), and a corticosteroid (e.g., fluticasone propionate or fluticasone furoate) in water to form a first dispersion, (iii) dispersing (and optionally homogenizing) a suspending agent (e.g., a co-spray dried combination of microcrystalline cellulose and carboxymethylcellulose sodium) in water to form a second dispersion, (iv) adding the first dispersion to the second dispersion to form a second active phase solution, (v) adding a chelating agent (e.g., disodium edetate such as disodium edetate dihydrate) and a buffering agent (e.g., monobasic sodium phosphate) to water to form a third solution, (vi) adding the first active phase solution and the third solution from step (v) to the second active phase solution (e.g., under stirring) to obtain a dispersion, (vii) adding a preservative (e.g., benzalkonium chloride) to the solution prepared in step (vi), (viii) optionally, adjusting the pH of the solution from step (vii) using sodium hydroxide (e.g., to a pH of 6.0 to 7.5, 6.5 to 7.0 or 6.3 to 7.3), and (ix) optionally, adding water (e.g., purified water) to the solution of step (viii) to obtain a desired volume and/or concentration for each component.

The final formulation may be characterized for droplet size distribution by Spraytec and particle size distribution by Copley.

Compositions prepared by the process as described herein can withstand the accelerated stability conditions of temperature and relative humidity and maintain their physical and chemical integrity at accelerated conditions of stability.

### Methods of Treatment

Another embodiment is a method of treating allergic rhinitis, allergic rhino-conjunctivitis, or symptoms thereof (such as nasal congestion) in a patient in need thereof by intranasally administering an effective amount of alcaftadine or a pharmaceutically acceptable salt thereof and a corticosteroid (such as a topical pharmaceutical composition comprising alcaftadine or a pharmaceutically acceptable salt thereof and a corticosteriod). Preferably, the topical pharmaceutical composition of the present invention is intranasally administered. In one embodiment, the patient suffers from allergic rhinitis. In another embodiment, the patient suffers from seasonal allergic rhinitis. In yet another embodiment, the patient suffers from perennial allergic rhinitis. In yet another embodiment, the patient suffers from moderate to severe seasonal allergic rhinitis. In yet another embodiment, the patient suffers from moderate to severe perennial allergic rhinitis. In one embodiment, the topical pharmaceutical composition is intranasally administered as 1 or 2 sprays per nostril of the patient once daily. In another embodiment, the topical pharmaceutical composition is intranasally administered as 1 or 2 sprays per nostril of the patient twice daily. Each spray of the topical pharmaceutical composition may comprise (i) about 171.25 mcg, about 342.5 mcg, about 685 mcg of alcaftadine and (ii) about 49.9 mcg of fluticasone propionate or about 27.4 mcg of fluticasone furoate.

The topical pharmaceutical composition of the present invention can be administered in the nostril(s) or to the eyes once or twice a day. In one embodiment, from about 340 to about 5500 mcg of alcaftadine and from about 80 to about 400 mcg of fluticasone propionate are administered daily (such as in one dose once daily, or in two equally divided doses twice daily). For instance, from about 170 to about 2740 mcg of alcaftadine and from about 40 to about 200 mcg of fluticasone propionate can be administered to each nostril daily. In another embodiment, from about 340 to about 5500 mcg of alcaftadine and from about 50 to about 250 mcg of fluticasone furoate are administered daily (such as in one dose once daily, or in two equally divided doses twice daily). For instance, from about 170 to about 2740 mcg of alcaftadine and from about 25 to about 125 mcg of fluticasone propionate can be administered to each nostril daily.

In one embodiment, one spray of a topical pharmaceutical composition containing alcaftadine and corticosteroid (such as described herein) is administered per nostril once per day.

In another embodiment, two sprays of a topical pharmaceutical composition containing alcaftadine and corticosteroid (such as described herein) are administered per nostril once per day.

In yet another embodiment, one spray of a topical pharmaceutical composition containing alcaftadine and corticosteroid (such as described herein) is administered per nostril twice per day.

In yet another embodiment, two sprays of the topical pharmaceutical composition described herein are administered per nostril twice per day.

Each spray of the topical pharmaceutical composition may provide from about 170 to about 685 mcg of alcaftadine (on a free base basis), such as about 171.25, 342.5, or 685 mcg of alcaftadine. Each spray of the topical pharmaceutical composition may provide 49 to 51 mcg of fluticasone propionate (such as about 49.9 mcg of fluticasone propionate). Each spray of the topical pharmaceutical composition may provide 26 to 29 mcg of fluticasone furoate (such as about 27.4 mcg of fluticasone furoate).

In one embodiment, from about 170 to about 1370 mcg alcaftadine and from about 40 to about 100 mcg of fluticasone propionate are administered once or twice daily to each nostril. In another embodiment, about 171.25, 342.5, 685, or 1370 mcg of alcaftadine and about 49.9 mcg of fluticasone propionate is administered to each nostril once daily. In yet another embodiment, about 171.25, 342.5, 685, or 1370 mcg of alcaftadine and about 49.9 mcg of fluticasone propionate is administered to each nostril twice daily.

In another embodiment, from about 170 to about 1370 mcg alcaftadine and from about 25 to about 60 mcg of fluticasone furoate are administered once or twice daily to each nostril. In another embodiment, about 171.25, 342.5, 685, or 1370 mcg of alcaftadine and about 27.4 mcg of fluticasone furoate is administered to each nostril once daily. In yet another embodiment, about 171.25, 342.5, 685, or 1370 mcg of alcaftadine and about 27.4 mcg of fluticasone propionate is administered to each nostril twice daily.

In one embodiment of the methods described herein, from about 340 to about 5480 mcg of alcaftadine and from about 90 to about 400 mcg of fluticasone propionate are administered daily to a patient in need thereof. In another embodiment, from about 340 to about 1370 mcg of alcaftadine and from about 90 to about 200 mcg of fluticasone propionate are administered daily to a patient in need thereof. In yet another embodiment, from about 340 to about 690 mcg of alcaftadine and from about 90 to about 200 mcg of fluticasone propionate are administered daily to a patient in need thereof.

In one embodiment of the methods described herein, from about 340 to about 5480 mcg of alcaftadine and from about 90 to about 420 mcg of fluticasone propionate are administered daily to a patient in need thereof. In another embodiment, from about 685 to about 2740 mcg of alcaftadine and from about 90 to about 210 mcg of fluticasone propionate are administered daily to a patient in need thereof. In yet another embodiment, from about 685 to about 1370 mcg of alcaftadine and from about 90 to about 210 mcg of fluticasone propionate are administered daily to a patient in need thereof. In yet another embodiment, from about 5000 to about 5480 mcg (e.g., 5280 mcg) of alcaftadine and from about 380 to about 420 mcg (e.g., 399.2 mcg) of fluticasone propionate are administered daily to a patient in need thereof.

In another embodiment of the methods described herein, from about 340 to about 5480 mcg of alcaftadine and from about 50 to about 220 mcg of fluticasone furoate are administered daily to a patient in need thereof. In another embodiment, from about 340 to about 1370 mcg of alcaftadine and from about 50 to about 110 mcg of fluticasone furoate are administered daily to a patient in need thereof. In yet another embodiment, from about 340 to about 690 mcg of alcaftadine and from about 50 to about 110 mcg of fluticasone furoate are administered daily to a patient in need thereof.

In one embodiment, the patient is 6 to 17 years of age. In another embodiment, the patient is 17 or 18 years of age or older.

### Definitions

By “pharmaceutically acceptable excipients”, it is meant any of the components of a pharmaceutical composition other than the active ingredients and which are approved by regulatory authorities or are generally regarded as safe for human or animal use.

As used herein, the term “mucoadhesion” refers to adhesion or adherence of a substance to a mucous membrane within the nasal mucosa. In the context of the present invention, the mucoadhesion is intended to convey a material that is capable of adhering to the nasal mucosa when placed in contact with that surface in order to enable compositions of the invention to adhere to that surface. Such materials are hereinafter referred to together as “mucoadhesives” or “mucoadhesive agents.”

The term “allergic rhinitis” includes allergic reactions of the nasal mucosa and includes hay fever, seasonal allergic rhinitis, and perennial rhinitis (non-seasonal allergic rhinitis) which are characterized by seasonal or perennial sneezing, rhinorrhea, nasal congestion, pruritus and eye itching, redness and tearing.

The term “patient” or “subject” refers to a human patient unless indicated otherwise. The patient can be 12 years of age or older or 18 years of age or older. The patient can also be 6 to 17 years of age.

The terms “treat,” “treatment,” and “treating” in the context of the administration of a therapy to a patient refers to the reduction or inhibition of the progression and/or duration of a disease or condition, the reduction or amelioration of the severity of a disease or condition, and/or the amelioration of one or more symptoms thereof resulting from the administration of one or more therapies.

An “effective amount” is an amount sufficient for a compound to accomplish a stated purpose relative to the absence of the compound (e.g. reduce one or more symptoms of a disease or condition). An example of an “effective amount” is an amount sufficient to contribute to the treatment, prevention, delay, inhibition, suppression, or reduction of a symptom or symptoms of a disease or disorder, which could also be referred to as a “therapeutically effective amount.” A “reduction” of a symptom or symptoms (and grammatical equivalents of this phrase) means decreasing of the severity or frequency of the symptom(s), or elimination of the symptom(s). An “effective amount” of a drug can be an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins). Dosages may be varied depending upon the requirements of the patient and the compound being employed. The dose administered to a patient, in the context of the present disclosure, should be sufficient to effect a beneficial therapeutic response in the patient over time. The size of the dose may also be determined by the existence, nature, and extent of any adverse side-effects. Determination of the proper dosage for a particular situation is within the skill of the practitioner.

As used herein, unless indicated otherwise, the term “stable” refers to a pharmaceutical composition of the present invention, which after 3 or 6 months storage at 25° C. and 60% relative humidity, 30° C. and 60% relative humidity, or 40° C. and 75% relative humidity has at least 90, 95, or 98% of the initial amount of each active ingredient present.

As used herein, the term “salts” or “pharmaceutically acceptable salt”, it is meant those salts, solvate and esters which are, within the scope of sound medical judgment, suitable for use in contact with the tissues of humans and lower animals without undue toxicity, irritation, and allergic response, commensurate with a reasonable benefit to risk ratio, and effective for their intended use. Representative acid additions salts include hydrochloride, furoate, hydrobromide, sulphate, bisulphate, acetate, oxalate, valerate, oleate, palmitate, stearate, laurate, borate, benzoate, lactate, phosphate, tosylate, mesylate, citrate, maleate, fumarate, succinate, tartrate, ascorbate, glucoheptonate, lactobionate, and lauryl sulphate salts. Representative alkali or alkaline earth metal salts include sodium, calcium, potassium and magnesium salts.

The transitional term “comprising,” which is synonymous with “including,” “containing,” or “characterized by,” is inclusive or open-ended and does not exclude additional, unrecited elements or method steps. The specification will be understood to also include embodiments which have the transitional phrase “consisting of” or “consisting essentially of” in place of the transitional phrase “comprising.” The transitional phrase “consisting of” excludes any element, step, or ingredient not specified in the claim, except for impurities associated therewith. The transitional phrase “consisting essentially of” limits the scope of a claim to the specified materials or steps “and those that do not materially affect the basic and novel characteristic(s)” of the claimed invention.

The present invention is further illustrated by reference to the following examples which is for illustrative purpose only and does not limit the scope of the invention in any way.

### Examples 1A and 1B

The aqueous compositions shown in Tables 1A below were prepared.

TABLE 1A

| | Example 1A | Example 1B |
|---|---|---|
| Components | (% w/w) | (% w/w) |
| Alcaftadine | 0.125 | 0.125 |
| Fluticasone Propionate | 0.03645 | — |
| Fluticasone Furoate | — | 0.02 |
| Sodium chloride | 0.68 | 0.68 |
| Monobasic sodium phosphate | 0.019 | 0.019 |
| Edetate Disodium, Dihydrate | 0.05 | 0.05 |
| Benzalkonium chloride (50%)** | 0.025 | 0.025 |
| Sodium hydroxide | 6.0-7.5 pH | 6.0-7.5 pH |
| Hydrochloric acid | 0.25 | 0.25 |
| Glycerine | 2.1 | 2.1 |
| Polysorbate 80 | 0.003 | 0.003 |
| Avicel CL611 | 3.64 | 3.64 |
| Purified water | Q.S. | Q.S. |

**Examples 1A and 1B include 0.0125% w/w of benzalkonium chloride since it is incorporated into the composition in the form of a 50% benzalkonium chloride solution.

Avicel CL611 (available from FMC Corporation of Philadelphia, Pa.) is a co-spray dried combination of microcrystalline cellulose and 11.3 to 18.8% sodium carboxymethylcellulose.

Examples 1A and 1B were prepared as follows:
a) Preparation of Alcaftadine Phase Solution: Hydrochloric acid, sodium chloride, and alcaftadine were added to a suitable quantity of purified water and stirred to obtain a clear Alcaftadine Phase Solution.
b) Preparation of Fluticasone Phase Solution: Polysorbate 80, glycerine, and fluticasone propionate or fluticasone furoate were added to a suitable quantity of purified water and homogenized to obtain a first uniform dispersion. Separately, microcrystalline cellulose and carboxymethylcellulose sodium was dispersed and homogenized in water to obtain a second uniform dispersion. The first uniform dispersion was added to the second uniform dispersion to obtain a Fluticasone Phase Solution.
c) Disodium edetate and monobasic sodium phosphate were added to the purified water, and stirred to obtain a clear Third Solution.
d) The Alcaftadine Phase Solution of step (a) and the Third Solution from step (c) were added to the Fluticasone Phase Solution of step (b) under stirring to obtain a uniform dispersion.
e) Benzalkonium chloride was added to the dispersion prepared in step (d) and stirred to obtain a uniform dispersion.
f) The pH of the dispersion prepared in step (e) was adjusted to 6.0-7.5 using sodium hydroxide (1M).
g) Purified water was added to the dispersion of step (f) to obtain the desired concentrations and volume.

Each spray of Examples 1A and 1B can provide 123.3 to 150.7 mg (e.g., 137 μL) of the composition, which would provide 171.25 mcg of alcaftadine for the compositions containing 0.125% w/w alcaftadine. Each spray of Example 1A provides 49.9 mcg of fluticasone propionate. Each spray of Example 1B provides 27.4 mcg of fluticasone furoate.

The stability of Examples 1A and 1B was assessed by measuring the amount of impurities, pH, viscosity, shot weight, and spray pattern initially and after 3 and 6 months (3M and 6M) storage at (a) 25° C. and 60% relative humidity (RH), (b) 30° C. and 75% RH, or (c) 40° C. and 75% RH. The results are shown in Table 1B-1G below.

TABLE 1B
Example 1A

| | | | Related Substances | | | | |
|---|---|---|---|---|---|---|---|
| | Related Substances | | Fluticasone propionate | | | | |
| | Alcaftadine (% w/w) | | (% w/w) | | | | |
| | Any | | Any | | | | Shot |
| Stability | Individual | Total | Individual | Total | | Viscosity | weight |
| condition | Impurity | Impurities | Impurity | Impurities | pH | (cPs) | (mg) |
| Initial | 0.076 | 0.126 | 0.135 | 0.32 | 6.74 | 131.42 | 138.52 |
| 3M, 25° C./ | 0.072 | 0.072 | ND | ND | 6.69 | 195.76 | 138.85 |
| 60% RH | | | | | | | |
| 3M, 30° C./ | 0.092 | 0.092 | ND | ND | 6.61 | 135.47 | 137.66 |
| 75% RH | | | | | | | |
| 3M, 40° C./ | 0.137 | 0.385 | ND | ND | 6.51 | 181.46 | 139.29 |
| 75% RH | | | | | | | |
| 6M, 25° C./ | ND | ND | 0.117 | 0.117 | 6.66 | 191.36 | 138.3 |
| 60% RH | | | | | | | |
| 6M, 30° C./ | 0.135 | 0.415 | ND | ND | 6.62 | 139.27 | 138.53 |
| 75% RH | | | | | | | |
| 6M, 40° C./ | 0.237 | 0.654 | 0.125 | 0.125 | 6.5 | 175.36 | 140.26 |
| 75% RH | | | | | | | |

ND = not determined

TABLE 1C
Droplet size distribution (Average): Example 1 A (at 3 cm)

| | D 10 | D 50 | D 90 | % <10 μm | SPAN |
|---|---|---|---|---|---|
| Initial | 16.65 | 35.4 | 77.29 | 0.61 | 1.71 |
| 3 M, 25° C./60% RH | 16.86 | 35.06 | 75.09 | 0.33 | 1.63 |
| 3 M, 30° C./75% RH | 16.47 | 33.17 | 72.31 | 0.42 | 1.67 |
| 3 M, 40° C./75% RH | 16.98 | 35.74 | 77.41 | 0.41 | 1.68 |
| 6 M, 25° C./60% RH | 16.77 | 35.55 | 78.6 | 0.42 | 1.73 |
| 6 M, 30° C./75% RH | 15.57 | 31.28 | 68.19 | 0.72 | 1.67 |
| 6 M, 40° C./75% RH | 18.29 | 40.64 | 88.88 | 0.37 | 1.73 |

TABLE 1D
Droplet size distribution (Average): Example 1A (at 6 cm)

| | D 10 | D 50 | D 90 | % <10 μm | SPAN |
|---|---|---|---|---|---|
| Initial | 19.84 | 35.2 | 62.46 | 0.8 | 1.21 |
| 3 M, 25° C./60% RH | 19.57 | 34.09 | 60.28 | 0.15 | 1.18 |
| 3 M, 30° C./75% RH | 19.53 | 33.66 | 58.67 | 0.26 | 1.08 |
| 3 M, 40° C./75% RH | 37.57 | 66.07 | 116.56 | 0.087 | 2.39 |
| 6 M, 25° C./60% RH | 19.32 | 34.44 | 61.84 | 0.12 | 1.23 |
| 6 M, 30° C./75% RH | 18.34 | 32.88 | 57.96 | 1.3 | 1.2 |
| 6 M, 40° C./75% RH | 19.6 | 34.96 | 62.19 | 0.99 | 1.21 |

TABLE 1E
Example 1B

| | | | Related Substances | | | | |
|---|---|---|---|---|---|---|---|
| | Related Substances | | Fluticasone furoate | | | | |
| | Alcaftadine (% w/w) | | (% w/w) | | | | |
| | Any | | Any | | | | Shot |
| Stability | Individual | Total | Individual | Total | | Viscosity | weight |
| condition | Impurity | Impurities | Impurity | Impurities | pH | (cPs) | (mg) |
| Initial | 0.817 | 2.013 | 0.127 | 0.415 | 7.43 | 151.47 | 139.95 |
| 3M, 25° C./ | 0.879 | 2.343 | 0.130 | 0.541 | 7.36 | 189.06 | 140.73 |
| 60% RH | | | | | | | |
| 3M, 30° C./ | 0.658 | 2.188 | 0.251 | 0.546 | 7.22 | 140.87 | 139.35 |
| 75% RH | | | | | | | |
| 3M, 40° C./ | 0.623 | 2.299 | 0.271 | 0.585 | 6.99 | 152.07 | 139.86 |
| 75% RH | | | | | | | |
| 6M, 25° C./ | 0.925 | 2.294 | 0.109 | 0.269 | 7.38 | 206.76 | 138.74 |
| 60% RH | | | | | | | |
| 6M, 30° C./ | 0.563 | 1.791 | 0.192 | 0.563 | 7.06 | 149.57 | 139.27 |
| 75% RH | | | | | | | |
| 6M, 40° C./ | 0.946 | 2.781 | 0.206 | 0.638 | 6.6 | 178.76 | 139.28 |
| 75% RH | | | | | | | |

TABLE 1F
Droplet size distribution (Average): Example 1B (at 3 cm)

| | D 10 | D 50 | D 90 | % <10 μm | SPAN |
|---|---|---|---|---|---|
| Initial | 17.13 | 39.07 | 88.98 | 0.6 | 1.83 |
| 3 M, 25° C./60% RH | 17.71 | 37.39 | 79.84 | 0.28 | 1.65 |
| 3 M, 30° C./75% RH | 17.13 | 38.47 | 87.67 | 0.51 | 1.83 |
| 3 M, 40° C./75% RH | 18.65 | 43.02 | 95.75 | 0.25 | 1.79 |
| 6 M, 25° C./60% RH | 18.37 | 40.04 | 84.35 | 0.71 | 1.64 |
| 6 M, 30° C./75% RH | 17.13 | 38.19 | 88.1 | 0.52 | 1.86 |
| 6 M, 40° C./75% RH | 17.38 | 37.07 | 77.92 | 0.6 | 1.62 |

TABLE 1G
Droplet size distribution (Average): Example 1B (at 6 cm)

| | D 10 | D 50 | D 90 | % <10 μm | SPAN |
|---|---|---|---|---|---|
| Initial | 17.64 | 34.61 | 67.67 | 2.1 | 1.44 |
| 3 M, 25° C./60% RH | 20.06 | 35.03 | 62.43 | 0.14 | 1.21 |
| 3 M, 30° C./75% RH | 18.48 | 34.34 | 66.69 | 0.304 | 1.38 |
| 3 M, 40° C./75% RH | 20.12 | 37.1 | 71.8 | 0.6 | 1.38 |
| 6 M, 25° C./60% RH | 19.25 | 35.83 | 66.75 | 1.15 | 1.32 |
| 6 M, 30° C./75% RH | 18.98 | 35.19 | 65.2 | 1.3 | 1.31 |
| 6 M, 40° C./75% RH | 19.17 | 34.76 | 63.51 | 1.16 | 1.26 |

### Example 2

The aqueous compositions 2A-2H shown in Tables 2A and 2B were prepared by the procedure described in Example 1.

TABLE 2A

| | 2A | 2B | 2C | 2D |
|---|---|---|---|---|
| Components | (% w/w) | (% w/w) | (% w/w) | (% w/w) |
| Alcaftadine | 0.05 to 5 | | | |
| Fluticasone furoate | 0.01 to 5 | 0.01 to 5 | — | — |
| Fluticasone propionate | — | — | 0.01 to 5 | 0.01 to 5 |
| Hydroxypropylmethyl | 0.01 to 1.0 | — | — | — |
| cellulose | | | | |
| Povidone | — | 0.01 to 1.0 | 0.01 to 1.0 | — |
| Sodium | — | — | — | 0.01 to 1.0 |
| carboxymethylcellulose | | | | |

| | |
|---|---|
| Sodium chloride | 0.068 to 6.80 |
| Monobasic sodium | 0.0019 to 0.19 |
| phosphate | |
| Edetate disodium, | 0.005 to 0.5 |
| dihydrate | |
| Benzalkonium chloride | 0.0025 to 0.25 |
| Phenylethyl alcohol | 0.025 to 2.5 |
| Polysorbate 80 | 0.001 to 5 |
| Microcrystalline | 0.5 to 15 |
| cellulose and | |
| carboxymethylcellulose | |
| sodium | |
| Sodium hydroxide | q.s. |
| Hydrochloric acid | 0.025 to 2.5 |
| Purified water | q.s. |

TABLE 2B

| | 2E | 2F | 2G | 2H |
|---|---|---|---|---|
| Components | (% w/w) | (% w/w) | (% w/w) | (% w/w) |
| Alcaftadine | 0.05 to 5 | | | |
| Fluticasone furoate | — | 0.01 to 5 | — | 0.01 to 5 |
| Fluticasone propionate | 0.01 to 5 | — | 0.01 to 5 | — |
| Hydroxypropylmethyl | — | — | 0.01 to 1.0 | — |
| cellulose | | | | |
| Sodium | — | — | — | 0.01 to 1.0 |
| carboxymethylcellulose | | | | |
| Hydroxyethyl | 0.01 to 1.0 | 0.01 to 1.0 | — | — |
| cellulose-L | | | | |

| | |
|---|---|
| Sodium chloride | 0.068-6.80 |
| Monobasic sodium | 0.0019-0.19 |
| phosphate | |
| Edetate disodium, | 0.005-0.5 |
| dihydrate | |
| Benzalkonium chloride | 0.0025-0.25 |
| Phenylethyl alcohol | 0.025-2.5 |
| Polysorbate 80 | 0.001 to 5% |
| Microcrystalline | 0.50-15 |
| cellulose and | |
| carboxymethylcellulose | |
| sodium | |
| Sodium hydroxide | q.s. |
| Hydrochloric acid | 0.025-2.5 |
| Purified water | q.s. |

### Example 3: Bitterness of Intranasal Alcaftadine

The formulation of alcaftadine shown in the table below was evaluated for bitterness and compared to a similar formulation without alcaftadine (placebo formulation) and three commercial intransal products (Astelin®, Patanase®, and Azep®).

| Ingredients | % w/w |
|---|---|
| Alcaftadine | 0.125 |
| Benzalkonium chloride | 0.025 |
| Disodium edetate | 0.050 |
| Monobasic sodium phosphate | 0.019 |
| Sodium chloride | 0.68 |
| Hypromellose 2910 | 0.1 |
| Hydrochloric acid | 0.25 |
| Sodium hydroxide | q.s. |
| Purified water | q.s. |

The alcaftadine formulation was prepared as follows:

a) Preparation of active phase solution: A suitable quantity of purified water and benzalkonium chloride were dissolved under stirring. Alcaftadine was added to the solution and stirred to get a uniform dispersion. Hydrochloric acid and sodium chloride was added to obtain a clear solution.

b) Preparation of bulk solution: A suitable quantity of purified water and hydroxypropylmethyl cellulose (Hypromellose 2910) was added and stirred to obtain a clear solution. Disodium edetate and monobasic sodium phosphate were added and stirred to obtain a clear solution.

c) Addition phase: The active phase solution prepared in step a) was added to the bulk solution prepared in step b) under stirring and a clear solution was obtained. The active phase solution container was rinsed with purified water and added to the bulk solution. The bulk solution was stirred and a clear solution was obtained.

d) pH adjustment: The pH of the solution prepared in step c) was adjusted to 6.0 to 8.0 using sodium hydroxide.

e) The volume of the batch was made using purified water.

The formulations were evaluated in 20 volunteers according to the evaluation criteria below.

| Evaluation Criteria | Score |
|---|---|
| Very much bitter | 4 |
| Bitter | 3 |
| Moderate bitter | 2 |
| No bitterness | 1 |

The results are shown in the table below.

| | | | Taste | |
|---|---|---|---|---|
| No. | Product name | Active | score | Evaluation |
| 1 | Alcaftadine | Alcaftadine | 1 | No bitterness |
| 2 | Placebo composition | None | 1 | No bitterness |
| | of the Alcaftadine | | | |
| | formulation | | | |
| 3 | Astelin ® | Azelastine | 4 | Very much |
| | | hydrochloride | | bitter |
| 4 | Patanase ® | Olopatadine | 2 | Moderately |
| | | hydrochloride | | bitter |
| 5 | Azep ® | Azelastine | 4 | Very much |
| | | hydrochloride | | bitter |

Alcaftadine was found to have a more acceptable taste compared to azelastine hydrochloride and olopatadine hydrochloride.

### Example 4: Pharmacokinetics of Intranasal Alcaftadine

The nasal formulations of Examples 4A, 4B and 4C shown below were prepared by a process as described in Example 3 for the alcaftadine formulation.

| | Example 4A | Example 4B | Example 4C |
|---|---|---|---|
| Ingredients | (% w/w) | (% w/w) | (% w/w) |
| Alcaftadine | 0.125 | 0.25 | 0.50 |
| Benzalkonium chloride | 0.025 | 0.025 | 0.025 |
| Disodium edetate | 0.050 | 0.050 | 0.050 |
| Monobasic sodium phosphate | 0.019 | 0.019 | 0.019 |
| Sodium chloride | 0.68 | 0.68 | 0.68 |
| Hypromellose 2910 | 0.1 | 0.1 | 0.1 |
| Hydrochloric acid | 0.25 | 0.25 | 0.25 |
| Sodium hydroxide | q.s. | q.s. | q.s. |
| Purified water | q.s. | q.s. | q.s. |

The pharmacokinetic profiles of the alcaftadine test formulations 4A, 4B, and 4C as well as an olapatdine nasal formulation were each determined on 6 male New Zealand white rabbits. The olopatadine nasal formulation (0.6% w/v) was prepared from Olopat Max® 0.7% w/v, where Olopat Max® was diluted to 0.6% w/v by using purified water.

The study was carried out on total 24 male New Zealand white rabbits, which were segregated into four groups (G1 to G4, 6 rabbits/group). Mild anaesthesia was induced in all rabbits by inhalation of 2% isoflurane. Three groups (G1-G3) of anaesthetized rabbits were intranasally administered 250 μL of alcaftadine once-daily using the formulations of Examples 10A, 10B, and 10C. A comparator, 250 μL of olopatadine at a concentration of 0.6% w/v, was intranasally administered once-daily to a fourth group G4 of anaesthetized rabbits.

Sampling details for the test and reference formulations: Approximately 0.5 mL of blood sample from each rabbit was withdrawn via marginal ear vein into pre-labelled Eppendorf tubes containing 10% EDTA as an anticoagulant. Blood samples were withdrawn at 0 min, 5 min, 10 min, 15 min, 30 min, 60 min, 120 min, 240 min, 480 min and 1440 min after administration of test and reference formulations.

Procedure for sample preparation: Collected blood samples were mixed gently and kept on crushed ice. Plasma samples were separated after centrifugation at 3500 rpm for 10 min at 4° C. Plasma separation was carried out within 30 min of sample collection and stored at −70±10° C. The animals were euthanized by intravenous administration of sodium thiopental injection overdose and nasal epithelium was collected. Half of the tissues of nasal epithelium were homogenized using phosphate buffered solution (20% homogenation) for quantification of alcaftadine and olopatidine. The remaining half of the tissues were stored in 10% neutral buffered formalin for detailed gross histopathological examination such as necrosis, inflammation or any changes during necropsy.

Rabbit plasma and nasal epithelial tissue concentrations of alcaftadine or olopatidine were determined using a fit-for-purpose LC-MS/MS method with LLOQ of 0.500 ng/mL or 0.250 ng/mL, respectively. The pharmacokinetic parameters were evaluated using Phoenix WinNonlin® Ent-Version 8.0 by non-compartmental analysis.

Results: The time to reach peak concentration following administration of 1.5 mg of olopatadine to New Zealand rabbits was 0.38 hours after a single administration. After a single intranasal instillation of alcaftadine at a doses of 0.312 mg, 0.625 mg and 1.25 mg, the time to achieve maximum concentration (Tₘₐₓ) was found to be 0.08 hours in all three groups. Tₘₐₓ was achieved much earlier than olopatdine at a dose of 1.5 mg. Thus, intranasal alcaftadine exhibited a faster onset of action than intranasal olopatadine.

After a single administration of alcaftadine at a doses of 0.625 to 1.25 mg, the area under the curve (AUC₀₋₂₄ₕ) and peak plasma concentrations (Cₘₐₓ) were dose proportional.

Following intranasal administration of alcaftadine at doses of 0.312 mg, 0.625 mg and 1.25 mg to rabbits, no signs or symptoms of inflammation or irritation were observed in any of the three groups. On histopathological examination, minimum to mild inflammatory cell (lymphocytes/heterophils) infiltration and increased mucous secretion in nasal epithelium was observed in a few animals across the groups administered the test and reference formulations.

The plasma concentration of alcaftadine was found to be dose proportional with overall low systemic exposure.

All publications, patents, and patent applications cited in this application are herein incorporated by reference to the same extent as if each individual publication, patent, or patent application was specifically and individually indicated to be incorporated herein by reference.

1. A topical composition for intranasal or ocular administration comprising alcaftadine or a pharmaceutically acceptable salt thereof and a corticosteroid or a pharmaceutically acceptable salt thereof.

2. The topical composition of claim 1, wherein the alcaftadine is in free form.

3. The topical composition of claim 1, wherein the corticosteroid is selected from alclometasone, beclometasone, betametasone, budesonide, ciclesonide, clobetasol, clobetasone, deflazacort, deprodone, dexamethasone, diflucortolone, fluocinolone, etiprednol, flunisolide, fluocinonide, fluocortolone, fluprednidene, flurometholone, fluticasone, halcinonide, hydrocortisone, loteprednol, methylprednisolone, mometasone, prednisolone, rimexolone, triamcinolone, esters thereof, and pharmaceutically acceptable salts thereof.

4. The topical composition of claim 1, wherein the corticosteroid is selected from beclomethasone, mometasone and esters thereof, fluticasone and esters thereof, budesonide, ciclesonide, and pharmaceutically acceptable salts thereof.

5. The topical composition of claim 1, wherein the corticosteroid is fluticasone furoate.

6. The topical composition of claim 1, wherein the corticosteroid is fluticasone propionate.

7. The topical composition of claim 1, further comprising a suspending agent.

8. The topical composition of claim 1, further comprising a preservative.

9. The topical composition of claim 1, further comprising at least 0.0125% w/w benzalkonium chloride, based upon the total weight of the composition.

10. The topical composition of claim 1, further comprising a buffering agent, a tonicity adjustment agent, a chelating agent, or any combination of any of the foregoing.

11. The topical composition of claim 10, wherein the buffering agent is monobasic sodium phosphate.

12. The topical composition of claim 10, wherein the tonicity adjustment agent is sodium chloride.

13. The topical composition of claim 10, wherein the chelating agent is disodium edetate.

14. An aqueous nasal pharmaceutical composition comprising (i) about 0.05 to about 5% w/w alcaftadine or a pharmaceutically acceptable salt thereof, (ii) about 0.01 to about 5% w/w fluticasone furoate or fluticasone propionate, (iii) optionally about 0.01 to about 1.0% w/w suspending agent, (iv) about 0.068 to about 6.8% w/w of sodium chloride, (v) about 0.0019 to about 0.19% w/w monobasic sodium phosphate, (v) about 0.005 to about 0.5% w/w disodium edetate, (vi) about 0.0025 to about 0.25% w/w benzalkonium chloride, (vii) about 0.001 to about 0.5% w/w polysorbate 80, and (viii) about 0.5 to about 15% w/w of a co-spray dried combination of microcrystalline cellulose and sodium carboxymethylcellulose, wherein the composition has a pH of 6.0 to 7.5.

15. An aqueous nasal pharmaceutical composition comprising (i) about 0.125% w/w alcaftadine, (ii) about 0.036% w/w fluticasone propionate or about 0.02% w/w fluticasone furoate, (iii) about 0.068 to about 6.8% w/w of sodium chloride, (iv) about 0.0019 to about 0.19% w/w monobasic sodium phosphate, (v) about 0.005 to about 0.5% w/w disodium edetate, (vi) about 0.0025 to about 0.25% w/w benzalkonium chloride, (vii) about 0.001 to about 0.5% w/w w/w polysorbate 80, and (viii) about 0.5 to about 15% w/w of a co-spray dried combination of microcrystalline cellulose and sodium carboxymethylcellulose, wherein the composition has a pH of 6.0 to 7.5.

16. An aqueous nasal pharmaceutical composition comprising (i) about 0.125% w/w alcaftadine, (ii) about 0.036% w/w fluticasone propionate or about 0.02% w/w fluticasone furoate, (iii) about 0.68% w/w of sodium chloride, (iv) about 0.019% w/w monobasic sodium phosphate, (v) about 0.05% w/w disodium edetate, (vi) about 0.0125% w/w benzalkonium chloride, (vii) about 2.1% glycerine, (viii) about 0.003% w/w polysorbate 80, and (ix) about 3.64% w/w of a co-spray dried combination of microcrystalline cellulose and sodium carboxymethylcellulose, wherein the composition has a pH of 6.0 to 7.5.

17. The topical composition of claim 1, wherein the composition has a viscosity of about 100 to about 400 cPs.

18. The topical composition of claim 1, wherein the pH of the composition is from about 6.0 to about 7.5.

19. The topical composition of claim 1 for use in the treatment of allergic rhinitis or one or more symptoms thereof.

20. The topical composition of claim 1 for use in the treatment of nasal congestion.

21. A method of treating allergic rhinitis or one or more symptoms thereof in a patient in need thereof comprising nasally administering to the patient an effective amount of the composition of claim 1.

22. A method of treating nasal congestion in a patient in need thereof comprising nasally administering to the patient an effective amount of the composition of claim 1.

23. A method of treating allergic rhinitis or one or more symptoms thereof in a patient in need thereof comprising nasally administering an effective amount of alcaftadine or a pharmaceutically acceptable salt thereof and a corticosteroid or a pharmaceutically acceptable salt thereof.

24. The method of claim 23, wherein the alcaftadine is in free form.

25. The method of claim 23, wherein the corticosteroid is selected from alclometasone, beclometasone, betametasone, budesonide, ciclesonide, clobetasol, clobetasone, deflazacort, deprodone, dexamethasone, diflucortolone, fluocinolone, etiprednol, flunisolide, fluocinonide, fluocortolone, fluprednidene, flurometholone, fluticasone, halcinonide, hydrocortisone, loteprednol, methylprednisolone, mometasone, prednisolone, rimexolone, triamcinolone, esters thereof, and pharmaceutically acceptable salts thereof.

26. The method of claim 23, wherein the corticosteroid is selected from beclomethasone, mometasone and esters thereof, fluticasone and esters thereof, budesonide, ciclesonide, and pharmaceutically acceptable salts thereof.

27. The method of claim 23, wherein the corticosteroid is fluticasone furoate.

28. The method of claim 23, wherein the corticosteroid is fluticasone propionate.
